(19) United States
(12) Patent Application Publication
(10) Pub. No.: US 2006/0161241 A1
(43) Pub. Date: Jul. 20, 2006

(54) Methods and devices for treating aortic atheroma

(76) Inventors: Denise Barbut, New York, NY (US); Mark Reisman, Kirkland, WA (US)

Correspondence Address: O'MELVENY & MYERS LLP, 610 NEWPORT CENTER DRIVE, 17TH FLOOR, NEWPORT BEACH, CA, 92660, US

(21) Appl. No.: 11/035,901

(22) Filed: Jan. 14, 2005

(51) Int. Cl. A61F 2/06

(52) U.S. Cl. 623/1.15

(57) ABSTRACT

A method for treating both sessile and mobile aortic atheroma is described. A radially expanding device, such as a stent or compliant cast, comprising a generally cylindrical member expandable between a compressed state and an enlarged state is provided. The cylindrical member has a proximal opening, a distal opening, a lumen therebetween, and at least one side opening in the wall of the generally cylindrical member. The methods comprise imaging the aorta to identify position and extent of atheroma. The stent is then advanced into the aortic arch and positioned so that the at least one side-opening is aligned with the takeoff of one or more of the right brachiocephalic artery, the left common carotid artery, or the left subclavian artery. The stent is expanded into contact with the endoluminal surface of the aorta and atheroma is trapped between the stent and the endoluminal surface of the aorta.

### FIELD OF THE INVENTION

The invention relates to devices and methods for treating mobile aortic atheroma, and more particularly to stents for trapping mobile aortic atheroma, as well as any of Grades 1, 2, 3, 4, or 5 plaque, against the endoluminal surface of the aorta.

### BACKGROUND

Atherosclerosis in the aorta can occur in patients as young as age 18. The atherosclerotic process may involve different parts of the aorta, such as the ascending aorta, the aortic arch, and the descending aorta, simultaneously or over a period of time. Aortic atherosclerosis may also occur concomitantly, precede, or follow carotid and or coronary atherosclerosis. Ascending and arch atherosclerosis is especially a recognized cause of cerebral vascular events, of which there are more than 2 million per year, and of problem during invasive aortic procedures such as cardiac catheterization or cardiac surgery. It is the most important risk factor for perioperative stroke.

Aortic atherosclerosis has been strongly associated with clinical embolic events, especially in the elderly patients. The atherosclerotic plaque can take on different morphologic features, including having mobile, ulceration, or protuberant components. Embolic risk appears to vary with different plaque types. Protuberant but stationary plaques, when located in the proximal aorta, are associated with an increased risk of embolization. Plaques with an ulcerated appearance or hypoechoic by ultrasound or calcified may also predispose a patient to develop significant embolic events. However, plaques with mobile components appear to have the highest embolic risk. The emboli can travel to the brain causing stroke, travel to the renal vasculature causing renal infarction, or travel to the distal extremities causing arm or leg ischemia.

To date, there is no good method for removing mobile plaque in the aorta. One way of removing mobile plaque would be to atherectomize. However, disadvantages of using such a method include (1) difficulty in localizing the mobile plaque and (2) risk of producing a shower of emboli during the procedure. Also, this would not address the treatment of non-mobile plaque, which is 5-10 times as common as mobile plaque. Therefore, new devices and methods are needed to treat both mobile and non-mobile aortic plaques that are at high risk of causing distal embolization to vital organs, including Grade 4 and 5 plaques.

### SUMMARY OF THE INVENTION

The invention provides methods for treating mobile aortic atheroma located in the ascending aorta, the aortic arch, and/or the descending aorta. The method involves the usage of a stent-like compliant cast comprising a generally cylindrical member or curved cylindrical member expandable between a compressed state that allows the stent to be advanced through narrow vessels and through the aorta and an enlarged state. In the enlarged state, the stent will engage the endoluminal surface of the aorta and thereby traps atheroma between the stent and the aorta. The stent has a proximal opening, a distal opening, and lumen therebetween, and may have at least one side opening in the wall of the generally cylindrical member or capabilities for making the opening after positioning to accommodate anatomic variation regarding positioning of the arch vessels.

In other cases, a mosaic stent will be used for making the opening after positioning to accommodate anatomic variation regarding positioning of the arch vessels. The stent could be of a self-expanding superelastic material, e.g., Nitinol stent to maintain aortic compliance or a braided stainless steel stent. The stent would likely endothelialize within days-weeks of placement. To accommodate the openings of the cerebral vessels, e.g., the brachiocephalic, left common carotid artery, or left subclavian artery, the stent might contain patches of a non-elastic material, e.g., steel with a looser metallic grid or strut or superheated nitinol. A wire would be advanced through each of the one or more patches to locate the cerebral take-offs and a balloon would then be used to dilate an orifice that would align with and allow blood flow into branching vessels. The orifices in some cases will be present prior to stent deployment, in other cases will be made during or after deployment.

In other cases, a modular stent will be used to accommodate anatomic variation regarding positioning of the arch vessels. The modular stent would, in some cases, consist of two separate components: a stainless steel module and a nitinol module. The stainless steel module would be placed inside the nitinol module. The nitinol module would have a large predetermined orifice wider than required for the cerebral take-off. The stainless steel stent would be inserted through this and positioned over the take-off and the orifice established using a balloon expander as described above for the mosaic stent. This orifice would more closely approximate the true diameter of the cerebral take-off.

The methods involve imaging the aorta to identify the atheroma and to determine its location. Any one of a number of imaging techniques can be used, including transthoracic echocardiography, transesophageal echocardiography, intravascular echocardiography, computed tomography, and magnetic resonance imaging.

After the location of one or more atheromatous plaque has been determined, a catheter carrying a stent is advanced into the aortic arch. The catheter may be entered through an incision in the femoral artery, the subclavian artery, the brachial artery, and the common carotid artery. The stent is positioned in the aortic arch so that the one or more side openings are aligned with the takeoff of one or more of the right brachiocephalic artery, left common carotid artery, and the left subclavian artery. The stent is also positioned so that it extends to cover one or more atheroma or an area affected by diffuse atheroma.

The stent is positioned using a catheter. The stent can be mounted on a balloon catheter so that the stent is deployed by inflating the balloon. Alternatively, the stent may be composed of a shaped memory material, e.g., super-elastic nitinol or other super-elastic material. In this alternative, the catheter need not carry a balloon but instead includes a mechanism for releasing the stent. The stent may also be introduced over the wire containing a distal protection mechanism attached.

After positioning, the stent is expanded into contact with the endoluminal surface of the aorta. Distal protection may be deployed before opening the stent. The atheroma is trapped between the stent and the endoluminal surface of the aorta. Once trapped, the plaque located on the portion of the atheromatous aortic wall behind the stent is unlikely to break free and cause distal embolization leading to, e.g., stroke, renal failure, mesenteric or spinal ischemia and ischemia of the distal extremities.

Depending on the region of placement, the cylindrical stent may have one side opening to engage the right brachiocephalic artery or the left subclavian artery. In other cases, the stent will have two side openings, one each for the right brachiocephalic artery and the left common carotid artery or for the left common carotid artery and the left subclavian artery. In still other cases where the stent will span all three of the great vessels, the stent will have three side openings or one large side opening which allows blood flow to all three of the great vessels. The stent may further be equipped with one or more sleeves that enter one or more great vessels. The stent may also be a drug eluting stent containing a drug such as sirolimus, tacrolimus, everolimus, and paclitxel.

In cases where a superelastic stent with patches of non-elastic material is used, the stent would first be partially opened in the aorta. A wire would first be passed through the patch into each cerebral take-off and a balloon would then be passed through the non-elastic material at the level of the cerebral vessel take-offs. The balloon is expanded to create holes wider than the vessel diameter. For example, if the brachiocephalic is 10 mm, the balloon would be inflated to make an orifice of 13-15 mm. It is generally desired to create an orifice that is 20% to 80% larger than the diameter of the branching vessel, in other cases 30% to 50% larger than the diameter of the branching vessel. Distal protection in the brachiocephalic, left common carotid artery, or left subclavian artery can optionally be used during this part of the procedure. Lumens would be present within the catheter to accommodate balloons and/or filters for each cerebral take-off. Then the balloons would be removed and the stent fully deployed with careful positioning of the orifices created to prevent obstruction of the take-offs.

Although the deployment of the stent is intended to protect the patient against embolization, it will understood that the positioning and deployment of the stent may, in certain cases, cause detachment of atheroma that could escape before the stent is fully expanded. Thus, the methods of the invention contemplate the use of distal protection devices downstream of the stent. In one case, the distal protection device is a filter. The filter may be place in the aorta downstream of the stent and preferably upstream of branching vessels, including the great arteries. In other cases, one or more filters will be placed in one or more of the right brachiocephalic artery, the left common carotid artery, and the left subclavian artery.

In other cases, the distal protection device is an occlusion balloon that causes flow reversal from a branching artery that has a source of collateral blood flow. The balloon may be placed in the right brachiocephalic artery downstream of the stent and inflated to at least partially obstruct the right brachiocephalic artery to cause flow reversal in a manner described in Barbut, U.S. Pat. Nos. 6,623,471, 6,595,980, 6,533,800, and 6,146,370, all incorporated herein by reference in their entirety. One or more additional occlusion balloons can be placed in the left common carotid artery and/or the left subclavian artery. In this way, any one, two, or all three of the right brachiocephalic artery, the left common carotid artery, and the left subclavian artery may receive an occlusion balloon or distal protection.

Moreover, a combination of distal protection filter and one or more occlusion balloons can be used together in the same procedure. Where distal protection devices are used, the one or more distal protection devices can be advanced into any of the great arteries in a retrograde direction from the right subclavian artery, the left common carotid artery, and or the left subclavian artery. Alternatively, the distal protection devices may be advanced in an antegrade direction from the aorta into any of the great arteries using a point of access on the femoral artery.

In another method, a first guidewire is passed through the subclavian artery of the arm, into the aorta, down the aorta, and out of a femoral sheath. A distal protection balloon occluder is then be passed over this first guidewire through the arm into the brachiocephalic takeoff (in the case of the right subclavian) or into the left subclavian takeoff (in the case of the left subclavian) and inflated partially or fully for distal protection. A second guidewire is advanced through the femoral artery up the leg, though the descending aorta, into the ascending aorta, and in certain procedures to the aortic valve.

The stent catheter is then prepared for entry into the aorta. The stent catheter is placed over the second guidewire that extends from the femoral artery to near the aortic valve so that the second guidewire extends through the central lumen of the stent. The distal end of the first guidewire is passed through the side opening in the stent. The stent is then advanced through the femoral artery, with the second guidewire going through the central lumen and the first guidewire going through the side opening in the stent. This allows good positioning of the stent over the orifice. When the stent reaches the aortic arch, the first guidewire aligns the side opening of the stent with the branching brachiocephalic artery while the stent expands.

In the preferred deployment technique, the stent is inserted transfemorally in a sheath. The sheath is positioned in the ascending aorta and the distal end anchored by a wire passed into the left ventricle. The sheath is then sequentially retracted, stopping beyond the right brachiocephalic orifice. At this point a wire is passed through one of the lumina through the stent into the cerebral takeoff (i.e., the right brachiocephalic artery, left common carotid artery, or the left subclavian artery) to locate the stent and then a balloon is inflated (introduced through the aorta or the arm) and the stent dilated to match the cerebral orifice. Then the rest of the sheath is retracted and the process repeated until the stent is dilated to account for all orifices.

### BRIEF DESCRIPTION OF THE DRAWINGS

FIG. 1A depicts a stent in accordance with the present invention.

FIG. 1B depicts a curved stent in accordance with the present invention.

FIG. 1C depicts a stent with a side opening in accordance with the present invention.

FIG. 2A depicts a stent having three side openings and three sleeves in accordance with the present invention.

FIG. 2B depicts a stent having an elongated side opening.

FIG. 2C depicts a stent having an elongated side opening and a side opening with a sleeve in accordance with the present invention.

FIG. 2D depicts a stent with a side opening having a sleeve.

FIG. 3A depicts a step in the deployment of a stent according to FIGS. 1A or 1B to treat mobile aortic atheroma in the ascending aorta.

FIG. 3B depicts a later step in the deployment of a stent to treat mobile aortic atheroma in the ascending aorta.

FIG. 4A depicts a step in the deployment of a stent to treat mobile aortic atheroma in the ascending aorta and in the aortic arch.

FIG. 4B depicts a later step in the deployment of a stent to treat mobile aortic atheroma in the ascending aorta and in the aortic arch.

FIG. 4C depicts a fully expanded stent in accordance with FIGS. 4A and 4B.

FIG. 5 depicts the stent of FIG. 2D deployed in the ascending aorta and in the aortic arch with occlusive protection in the brachiocephalic artery and filter protection downstream in the aortic arch.

FIG. 6 depicts the stent of FIG. 2A deployed in the ascending aorta and in the aortic arch with occlusive protection in the brachiocephalic, left common carotid artery, and the left subclavian artery and filter protection downstream in the aorta.

FIG. 7 depicts a step in the deployment of the stent of FIG. 2B deployed in the ascending aorta and in the aortic arch with occlusive protection in the left common carotid artery and the left subclavian artery and filter protection in the brachiocephalic artery and downstream in the aorta.

FIG. 8A depicts the stent of FIG. 2B being deployed in the ascending aorta, in the aortic arch, and in the descending aorta with filter protection in the brachiocephalic artery, the left common carotid artery, and the left subclavian artery.

FIG. 8B depicts a further step in deployment of the stent of FIG. 2B in the ascending aorta, in the aortic arch, and in the descending aorta with filter protection in the brachiocephalic artery, the left common carotid artery, and the left subclavian artery.

FIG. 9 depicts a stent with three side openings for use in the aortic arch.

FIG. 9A depicts the stent of FIG. 9 deployed in the ascending aorta, in the aortic arch, and in the descending aorta with filter protection in the brachiocephalic artery, the left common carotid artery, the left subclavian artery, and downstream in the aorta.

FIG. 10A depicts a step of the stent of FIG. 2B being deployed in the ascending aorta, in the aortic arch, and in the descending aorta with an elongated filter protecting the brachiocephalic artery, the left common carotid artery, and the left subclavian artery.

FIG. 10B depicts the stent of FIG. 2B deployed in the ascending aorta, in the aortic arch, and in the descending aorta with an elongated filter protecting the brachiocephalic artery, the left common carotid artery, and the left subclavian artery.

FIG. 11 depicts a stent deployed in the ascending aorta with downstream filter protection and a stent deployed in the abdominal aorta with downstream filter protection.

FIG. 12 depicts a first stent having side openings for the renal arteries deployed in the abdominal aorta with downstream filter protection and a second stent having a plurality of side openings for the spinal arteries deployed in the abdominal aorta.

FIG. 13 depicts a stent having side openings for the renal arteries and having a plurality of side openings for the spinal arteries deployed in the abdominal aorta with downstream filter protection.

### DETAILED DESCRIPTION

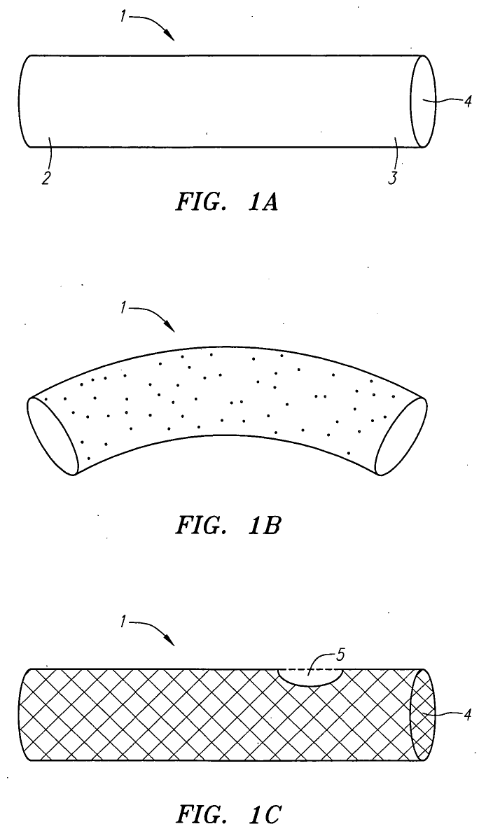

A first embodiment of an aortic stent for trapping plaque is shown in FIG. 1A. Stent 1 comprises an elongated cylindrical member having a first end 2, a second end 3, and a lumen 4 therebetween. The stent can be made of nitinol or stainless steel, or any other suitable material known in the art. The stent is expandable between a compressed state that allows the stent to be advanced through narrow vessels and through the aorta and an enlarged state. The stent can be generally straight as depicted in FIG. 1A or curved as depicted in FIG. 1B. The stent may have one or more side openings 5 as depicted in FIG. 1C to allow blood to flow into branching arteries. The stent can have small pores (FIG. 1B), no pores (FIG. 1A), or a mesh with large pores (FIG. 1C).

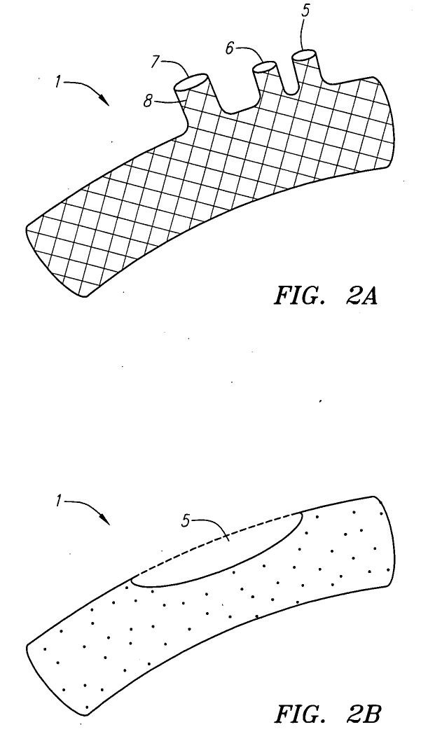

In another embodiment, the stent will include one, two, or three side openings as depicted in FIG. 2A. The one or more side openings may, in certain cases, be equipped with sleeves 8 that ensure proper alignment with vessels that branch from the aorta. In other cases, as shown in FIG. 2B, the stent contains an elongate side opening 5 that will allow blood flow to pass to a number of branching vessels. In still another embodiment, the stent will include both an elongate side opening 5 that allows blood flow to a number of vessels and smaller opening 7, with or without sleeve 8, as depicted in FIG. 2C. In other cases, as shown in FIG. 2D, stent 1 will have one opening 5 with sleeve 8.

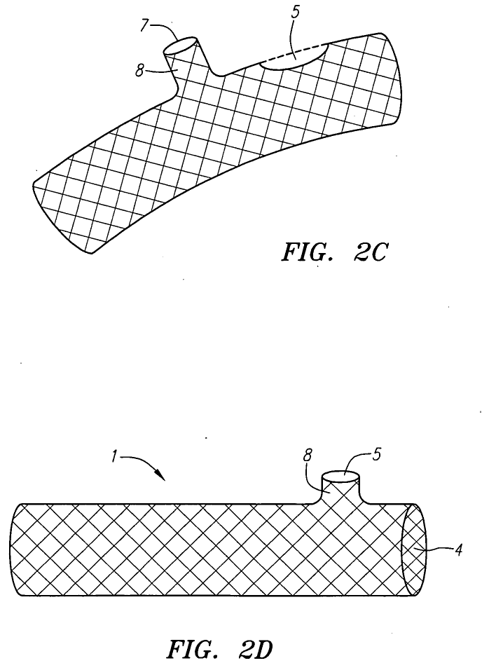

In use, the stent may be deployed in the ascending aorta, the aortic arch, the descending aorta, or the abdominal aorta to trap mobile aortic atheroma against the endoluminal wall of the aorta and thereby prevent downstream embolic events, e.g., stroke, renal infarction, or distal extremity infarction. The stent can be placed using a catheter or guidewire, with or without a filter, as depicted in FIG. 3A. Stent 1 is positioned on wire 20, and advanced into the ascending aorta adjacent mobile atheroma 99. Wire 20 may include a filter 25 proximal and downstream stent 1 to capture emboli dislodged during the procedure. When stent 1 is positioned, filter 25 is expanded to cover the endoluminal circumference of the aorta. The stent is then expanded, either by inflating a balloon or by release of a self-expanding stent. When the stent reaches and makes contact with the endoluminal wall, mobile aortic atheroma is trapped and held in place against the endoluminal surface of the aorta as depicted in FIG. 3B.

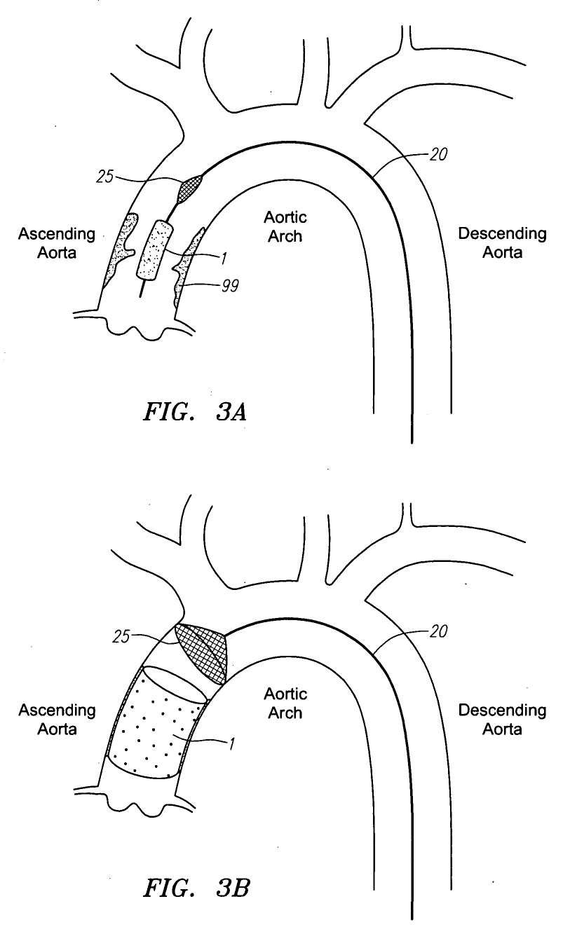

In another method of use, stent 1 is positioned in the ascending aorta and extends into the aortic arch as shown in FIG. 4A. The filter is secured to guidewire 20, which has distal filter 26 positioned in brachiocephalic artery 101 for protection of the cerebral vasculature, and proximal filter 25 positioned in the aorta downstream of stent 1 for protection of the other cerebral arteries and renal arteries. Guidewire 20 extends through side opening 5 on stent 1. With this arrangement, side opening 5 aligns with the takeoff of brachiocephalic artery 101 when deployed. FIG. 4B shows an alternative wherein filter 26, which protects brachiocephalic artery 101, is advanced separately, on wire 29, via the right subclavian or right brachial artery. The deployment of stent 1 is shown in FIG. 4C, wherein opening 5 communicates with brachiocephalic artery 101. After deployment, filter 26 is contracted and withdrawn and filter 25 is likewise contracted and withdrawn.

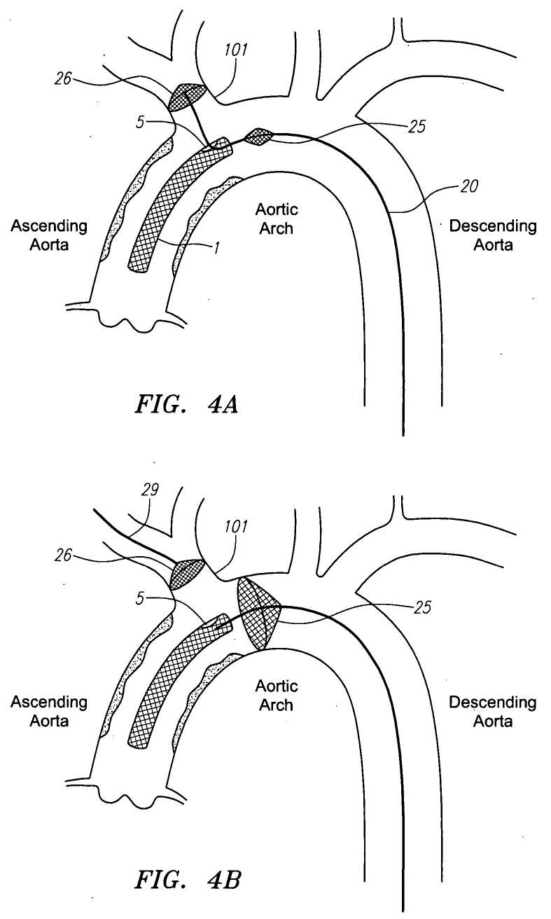

FIG. 5 shows a further method for deploying an aortic stent. Occlusion balloon 30 is positioned in brachiocephalic artery 101 held by elongate tubular member 31 inserted via right subclavian or right brachial artery. Balloon 30 is expanded causing blood flow to reverse and flow retrograde down the right internal carotid artery and right common carotid artery 104 and into right subclavian artery 105. A first filter 25 is deployed in the aortic arch between the brachiocephalic artery and left common carotid artery 102. A second filter 27 is deployed covering the takeoffs of left common carotid artery 102 and left subclavian artery 103. First filter 25 and second filter 27 are carried by guidewire 20, which also carries aortic stent 1. With one or both filters deployed and occlusion balloon 30 expanded, stent 1 is expanded into contact with the endoluminal surface of the aorta to trap mobile aortic atheroma. Side opening 5 is aligned to communicate with brachiocephalic artery 101.

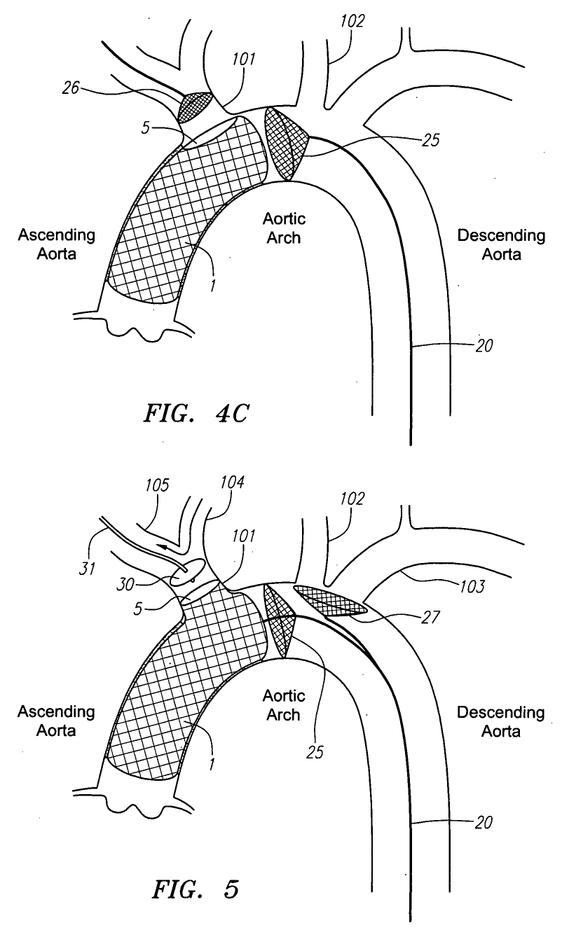

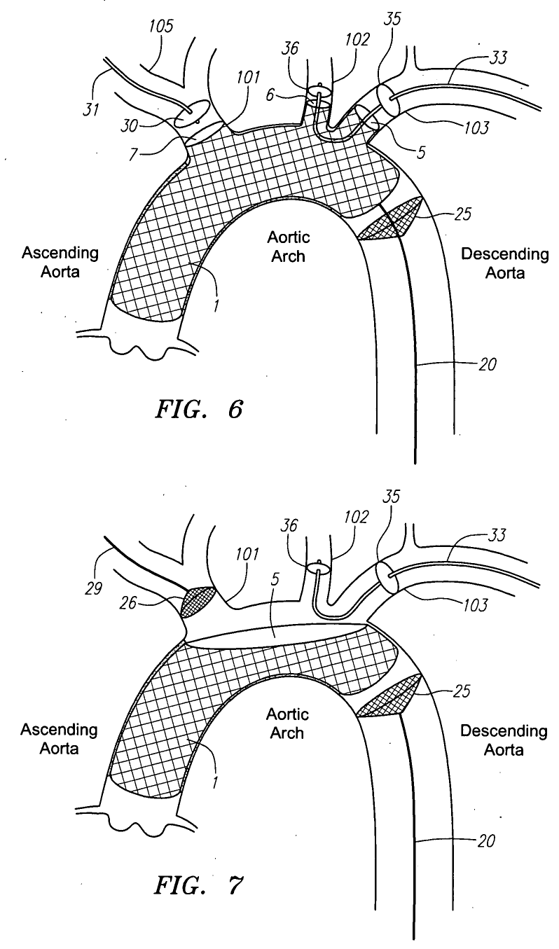

In FIG. 6, a stent is deployed having three separate side openings, each having a sleeve adapted to fit the takeoff of brachiocephalic artery 101, left common artery 102, and left subclavian artery 103, respectively. Occlusion balloon 30, mounted on elongate tubular member 31, protects the brachiocephalic artery. Tubular member 31 is inserted via the right subclavian artery or right brachial artery. Occlusion balloons 35 and 36, mounted on elongate tubular member 33 are inserted via the left subclavian artery. Elongate tubular member 33 extends through opening 5 of stent 1 and passes through opening 6 to access common carotid artery 102. Balloon 36 is located and expanded in the left common carotid artery while balloon 35 expands and protects left subclavian artery 103. Filter 25 carried by guidewire 20 is deployed downstream of the aortic stent to capture emboli inadvertently dislodged during stent deployment. With distal protection in place, stent 1 is expanded to trap mobile aortic atheroma against the endoluminal surface of the aorta. FIG. 7 depicts an alternative using a stent having elongate side opening 5 that extends from a position upstream the takeoff brachiocephalic artery 101 to a position downstream of left subclavian artery 103. This elongate opening allows each of the great arteries to communicate with blood flowing through the interior lumen of stent 1. In certain cases, the occlusion balloon in the brachiocephalic artery will be replaced by filter 26 deployed on guidewire 29 via right subclavian artery or right brachial artery.

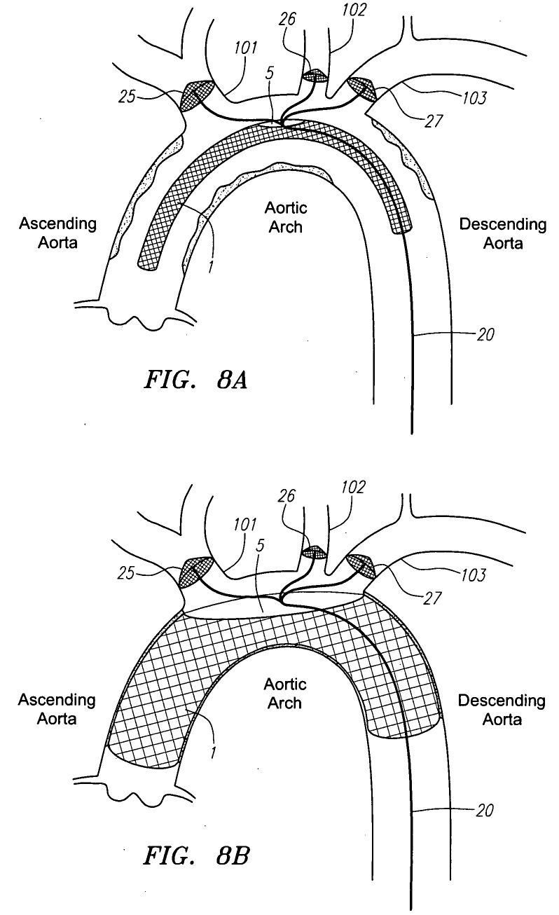

A further embodiment of a stent with distal protection is shown in FIG. 8A. Stent 1 includes elongate side opening 5, which aligns with the great arteries. Guidewire 20 carries three filters 25, 26, and 27, for placement in each of brachiocephalic artery 101, left common artery 102, and left subclavian artery 103, respectively. After the filters are in place and expanded, stent 1 is expanded to trap mobile aortic atheroma as shown in FIG. 8B. Guidewire 20 extends through opening 5 of stent 1 to access the great vessels. The passage of guidewire 20 through opening 5 helps to align opening 5 with the great vessels on expansion of the aortic stent.

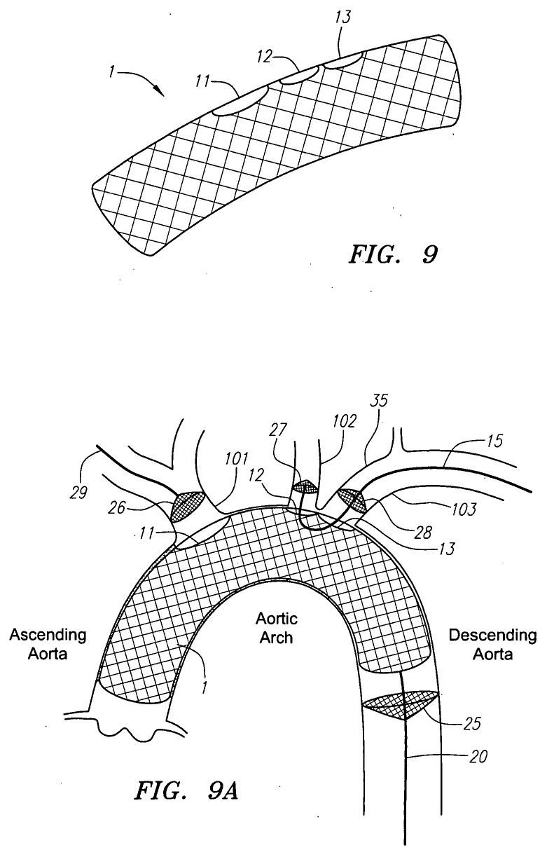

FIG. 9 depicts a mesh stent having three side openings 11, 12, and 13. The use of this stent with distal protection is shown in FIG. 9A. Stent 1 is carried at the distal end of guidewire 20, which also carries aortic filter 25. Filter 26, carried by guidewire 29, is located and expanded to protect brachiocephalic artery 101. Guidewire 29 is inserted through the right subclavian artery or the right brachial artery. Guidewire 15 , carrying first filter 27 and second filter 28, is inserted through the left subclavian artery. Guidewire 15 extends through opening 13 of stent 1 and further extends through opening 12 of stent 1 to access common carotid artery 102. Filter 27 expands to protect left common carotid artery 102 while filter 28 expands to protect left subclavian artery 103. Aortic stent 1 is then deployed to trap mobile aortic atheroma. Filters 25, 26, 27, and 28 are contracted for removal of guidewires 15, 20, and 29.

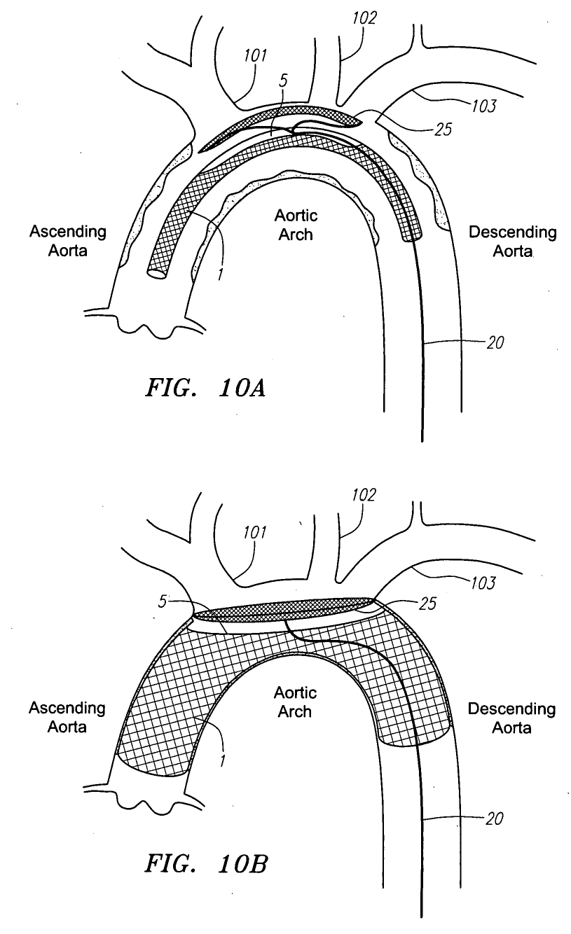

FIG. 10A shows the use of the aortic stent 1 having elongate side opening 5 with distal protection to cover all three great arteries at once. Filter 25 is attached to guidewire 20, which carries stent 1. Guidewire 20 extends through side opening 5 to allow placement of filter 25 over the takeoffs of the three great arteries. The extension of guidewire 20 through side opening 5 ensures the alignment of opening 5 with the great arteries. Stent 1 is expanded to trap mobile aortic atheroma as shown in FIG. 10B. Filter 25 is contracted and guidewire 20 and filter 25 are removed from the aorta.

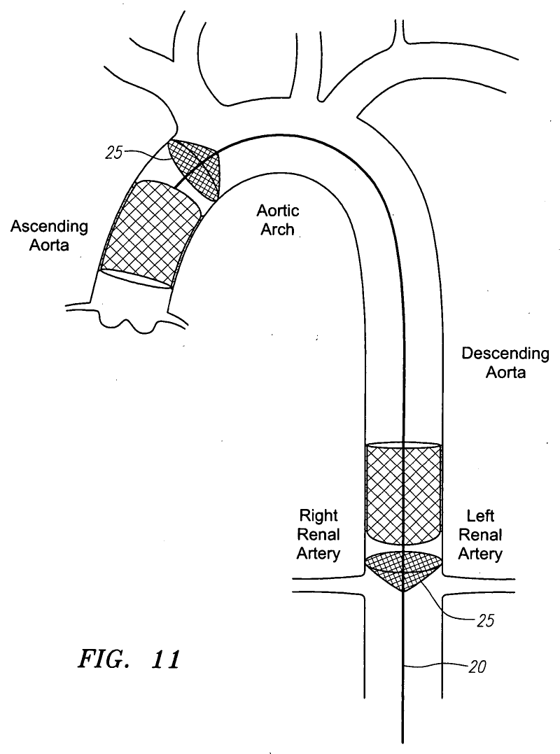

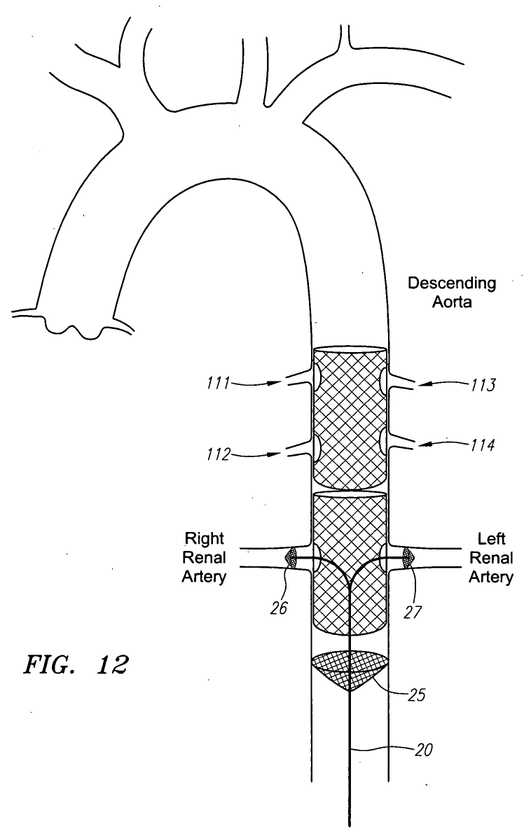

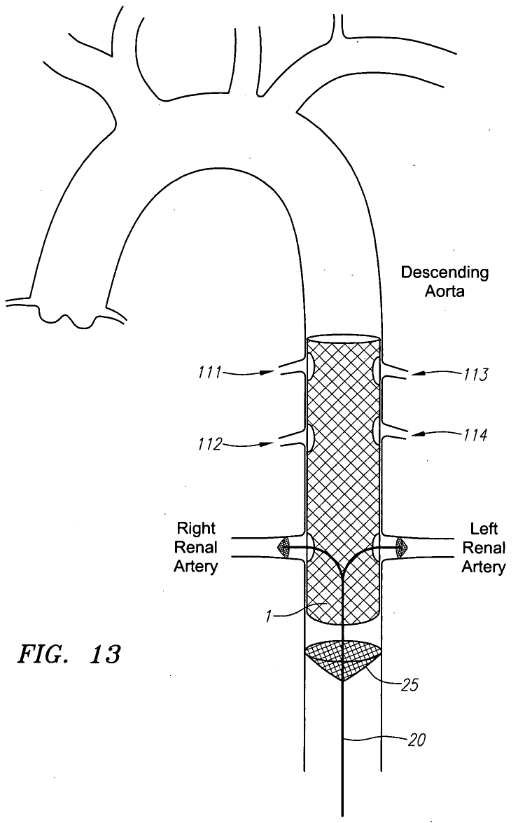

More than one stent may be placed in different areas of the aorta to trap mobile aortic atheroma. For example, FIG. 11 shows placement of a first stent in the ascending aorta with filter 26 providing distal protection. Filter 26 is deployed before expansion of the upstream stent. A second stent is deployed to trap mobile aortic atheroma in the abdominal region of the descending aorta. Filter 25 is deployed downstream of this second stent for protection of the renal arteries and the lower extremities. Both stents and filters are carried by guidewire 20. Alternatively, as depicted in FIG. 12, one or more stents may be placed in the region of superior mesenteric artery 111, inferior mesenteric artery 112, and spinal arteries 113 and 114. Such a stent will have one or more side openings such as shown in FIG. 12 to permit blood flow to these branching vessels. Moreover, a further stent can be placed in the region of the renal arteries as shown in FIG. 12. Guidewire 20 carries filter 25 for protection of the distal extremities, filter 26 for protection of the right renal artery and filter 27 for protection of the left renal artery. Filters 25, 26, and 27 are placed in their respective arteries and expanded before stent deployment. The aortic stent is expanded to trap mobile aortic plaque, the filters are contracted, and guidewire 20 with filters are removed.

In another embodiment, a single elongate stent can span a region from upstream superior mesenteric artery 111 to downstream of the renal arteries. Filter 25 and optional filters 26 and 27 are deployed respectively in the aorta, right renal artery, and the left renal artery. Stent 1 is expanded with side openings aligned to provide fluid communication between the branching arteries and blood flow through the lumen of stent 1. Filters 25 and optionally 26 and 27 are contracted and guidewire 20 is removed.

It should be understood that the devices and methods described herein can be used for the treatment of mobile aortic atheroma as well as the treatment of protuberant stationary plaques and ulcerated plaques in the aorta. Moreover, any of the various aortic stents can be used with any combination of filter protection and/or occlusive balloon protection.

The stents for use herein will generally range in length from 1 cm to 20 cm, in other cases from 3 cm to 15 cm, and in other cases from 5 cm to 8 cm. The stent will have a diameter before expansion of 1-10 mm, in other cases 2-8 mm, and in other cases 3-7 mm. After expansion, the stent will reach a diameter of 3-4 cm, in other cases 2-3 cm, and in other cases 1.5-2.5 cm depending on the location in the aorta and the anatomy of the individual patient. The foregoing ranges are intended only to illustrate typical device dimensions. Devices in accordance with the present invention can vary outside these ranges without departing from the inventive principles taught herein.

Although the foregoing invention has, for the purposes of clarity and understanding, been described in some detail by way of illustration and example, it will be obvious that certain changes and modifications may be practiced which will still fall within the scope of the appended claims. It will also be understood that any feature or features from any one embodiment, or any reference cited herein, may be used with any combination of features from any other embodiment.

What is claimed is:

1. A method for treating a mobile aortic atheroma, comprising the steps of:
providing a stent or compliant cast comprising a generally cylindrical member expandable between a compressed state and an enlarged state, the cylindrical member having a proximal opening, a distal opening, a lumen therebetween, and at least one side opening in the wall of the generally cylindrical member;
imaging the aorta to identify an atheroma;
advancing the stent into the aortic arch and positioning the stent so that the at least one side opening is aligned with the takeoff of one or more of the right brachiocephalic artery, the left common carotid artery, or the left subclavian artery and the stent covers the atheroma; and
expanding the stent into contact with the endoluminal surface of the aorta,
wherein the atheroma is trapped between the stent and the endoluminal surface of the aorta.

2. The method of claim 1, wherein the cylindrical member has two side openings.

3. The method of claim 1, wherein the cylindrical member has three side openings.

4. The method of claim 1, further comprising a sleeve extending from the at least one side opening and adapted to engage the endoluminal surface of one or more of the right brachiocephalic artery, the right common carotid artery, or the left subclavian artery.

5. The method of claim 1, wherein the stent is a self-expanding stent.

6. The method of claim 1, wherein the stent is mounted on a balloon and the stent is expanded by inflating the balloon.

7. The method of claim 1, wherein the stent is nitinol.

8. The method of claim 1, wherein the stent is a drug eluting stent including a drug selected from the group consisting of sirolimus, everolimus, tacrolimus, and paclitaxel.

9. The method of claim 1, wherein the step of imaging the aorta makes use of transthoracic echocardiogram, transesophageal echocardiogram, intravascular echocardiography, or magnetic resonance imaging.

10. The method of claim 1, further comprising the step of deploying a distal protection device before expanding the stent.

11. The method of claim 10, wherein the distal protection device is a filter.

12. The method of claim 11, wherein the filter is placed in the aorta downstream of the stent.

13. The method of claim 11, wherein the filter is placed in the right brachiocephalic artery downstream of the stent.

14. The method of claim 11, wherein the filter is placed in the left common carotid artery downstream of the stent.

15. The method of claim 11, wherein the filter is placed in the left subclavian artery downstream of the stent.

16. The method of claim 10, wherein the distal protection device is an occlusion balloon.

17. The method of claim 16, wherein the occlusion balloon is placed in the right brachiocephalic artery downstream of the stent, and inflated to at least partially obstruct the right brachiocephalic artery.

18-52. (canceled)

53. A method for treating a mobile aortic atheroma, comprising the steps of:
providing a stent or compliant cast comprising a generally cylindrical member expandable between a compressed state and an enlarged state, the cylindrical member having a proximal opening, a distal opening, and a lumen therebetween;
imaging the aorta to identify an atheroma;
advancing the stent into the aortic arch and positioning the stent so that the stent covers the atheroma;
expanding the stent into contact with the endoluminal surface of the aorta, wherein the atheroma is trapped between the stent and the endoluminal surface of the aorta; and
forming at least one side opening in the stent aligned with the takeoff of one or more of the right brachiocephalic artery, the left common carotid artery, or the left subclavian artery.

54. A method for treating a mobile aortic atheroma, comprising the steps of:
providing a stent or compliant cast comprising a generally cylindrical member expandable between a compressed state and an enlarged state, the cylindrical member having a proximal opening, a distal opening, and a lumen therebetween;
imaging the aorta to identify an atheroma;
advancing the stent into the aortic arch and positioning the stent so that the stent covers the atheroma;
forming at least one side opening in the stent aligned with the takeoff of one or more of the right brachiocephalic artery, the left common carotid artery, or the left subclavian artery; and
expanding the stent into contact with the endoluminal surface of the aorta, wherein the atheroma is trapped between the stent and the endoluminal surface of the aorta.

55-76. (canceled)
